(19) United States
(12) Patent Application Publication
(10) Pub. No.: US 2019/0111237 A1
(43) Pub. Date: Apr. 18, 2019

(54) CHANGING CATHETER INTO STEERING TOOL

(71) Applicant: Bendit Technologies Ltd., Petach Tikva (IL)

(72) Inventor: Oz Cabiri, Hod HaSharon (IL)

(73) Assignee: Bendit Technologies Ltd., Petach Tikva (IL)

(21) Appl. No.: 15/786,616

(22) Filed: Oct. 18, 2017

(51) Int. Cl. A61M 25/01; A61M 25/09

(52) U.S. Cl. CPC A61M 25/0133; A61M 25/09; A61B 2017/003; A61M 25/0113; A61M 25/0102

(57) ABSTRACT

A method of changing a catheter into a distal steering tool catheter includes adding to an existing catheter a tube assembly that passes through the catheter, the tube assembly including an internal tube and an external tube whose distal ends are coupled to each other and which are arranged for longitudinal axial movement relative to one another, and providing a tube manipulator coupled to the tube assembly, which is operative to cause relative axial movement of the internal and external tubes and bending of a distal portion of at least one of the internal and external tubes.

### FIELD OF THE INVENTION

The present invention generally relates to a method for changing a passive catheter into a catheter with a distal end steering tool and an assembly of a passive catheter and a distal end steering tool.

### BACKGROUND OF THE INVENTION

A typical prior art catheter 1 is shown in FIG. 1. Catheter 1 may have a proximal connection feature 2 (e.g., a luer lock or fluid connector). Catheter 1 includes a tube, which may have one or more segments, such as a proximal section 3, a distal section 4, and a distal tip 5, each of which has difference characteristics, such as but not limited to, dimensions, stiffness, added supportive structure, inner and outer coatings, and other features, such as a tip and radio pack.

Known catheters like catheter 1 are passive, that is, even though they may have soft distal ends, the distal end does not have the ability to bend through tortuous bends in body lumens.

### SUMMARY OF THE INVENTION

The present invention seeks to provide a method for changing a passive catheter into a catheter with a distal end steering tool and an assembly of a passive catheter and a distal end steering tool, as is described more in detail hereinbelow.

In the present invention, a steering tool tube assembly is added to the catheter and changes the catheter into a steering tool catheter. The steering tool tube assembly may include inner and outer tubes whose distal ends are coupled to each other. Alternatively, the steering tool tube assembly may include just an internal tube whose distal end is coupled to the distal end of the catheter.

Some non-limiting examples of commercially available catheters which may be modified by the methods and structure of the present invention into a steering tool catheter include:
SOFIA Distal Access Catheter by Microvention
CAT 6 Distal Access Catheter by Stryker
AXS Infinity LS Long Sheath by Stryker
FLOWGATE 2 balloon guide by Stryker
ACE family of reperfusion catheters by Penumbra
NEURON MAX System by Penumbra
ARC™ INTRACRANIAL SUPPORT CATHETER by Medtronic
FUBUKI guide catheter by Asahi

There is thus provided in accordance with an embodiment of the present invention a tube assembly that passes through a catheter, the tube assembly including an internal tube and an external tube whose distal ends are coupled to each other and which are arranged for longitudinal axial movement relative to one another, and a tube manipulator coupled to the tube assembly, which is operative to cause relative axial movement of the internal and external tubes and bending of a distal portion of at least one of the internal and external tubes.

In accordance with an embodiment of the present invention the external tube is different from the catheter. Both the internal and external tubes may pass through a distal end of the catheter or may abut against the distal end of the catheter.

In accordance with an embodiment of the present invention the external tube is the catheter.

In accordance with an embodiment of the present invention a stopper or any other coupling member is coupled to a distal end of the internal tube.

In accordance with an embodiment of the present invention the tube manipulator is operative to lock the tube assembly at a bent position.

There is thus provided in accordance with an embodiment of the present invention a method of changing a catheter into a distal steering tool catheter, including adding to an existing catheter a tube assembly that passes through the catheter, the tube assembly including an internal tube and an external tube whose distal ends are coupled to each other and which are arranged for longitudinal axial movement relative to one another, and providing a tube manipulator coupled to the tube assembly, which is operative to cause relative axial movement of the internal and external tubes and bending of a distal portion of at least one of the internal and external tubes.

### BRIEF DESCRIPTION OF THE DRAWINGS

The present invention will be understood and appreciated more fully from the following detailed description taken in conjunction with the drawings in which:

FIG. 1 is a simplified illustration of a prior art catheter;

FIG. 2 is a simplified illustration of an assembly of a passive catheter and a distal end steering tool, in accordance with a non-limiting embodiment of the present invention, in which the added steering tool is an assembly of an internal tube and an outer tube coupled to each other and which extends from the distal end of the existing catheter;

FIG. 3 is a simplified enlarged view of one end of the assembly of FIG. 2;

FIG. 4 is a simplified illustration of an internal tube with a stopper for coupling to the distal end of the existing catheter, in accordance with another non-limiting embodiment of the present invention;

FIG. 5 is a simplified illustration of the internal tube with stopper of FIG. 4 pre-assembled with a passive catheter, in accordance with another non-limiting embodiment of the present invention; and

FIG. 6 is a simplified illustration of the internal tube with stopper assembled and ready to use in the system.

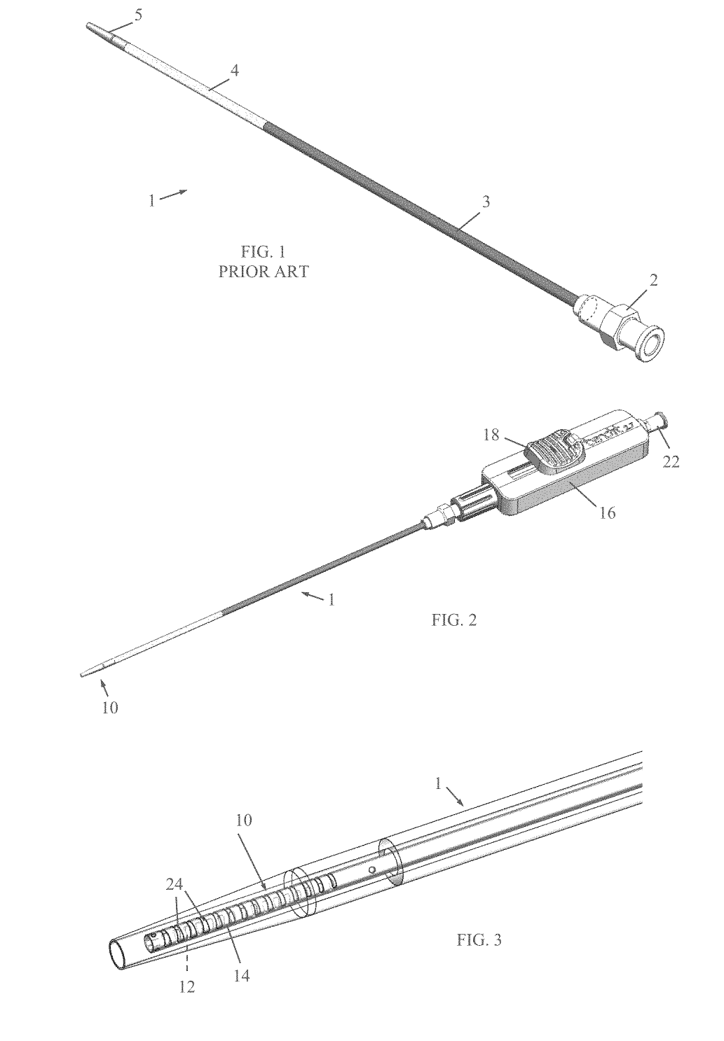

### DETAILED DESCRIPTION OF EMBODIMENTS

Reference is now made to FIG. 2, which illustrates an assembly of a passive catheter 1 and a distal end steering tool 10, in accordance with a non-limiting embodiment of the present invention, and to FIG. 3 which shows the structure of the steering tool 10.

As seen best in FIG. 3, steering tool 10 includes an internal tube 12 disposed inside an external tube 14. A distal end of internal tube 12 is fixedly joined to a distal end of external tube 14. The term “joined” encompasses any method for attaching the materials of the tubes together, such as but not limited to, welding, ultrasonic welding, thermal bonding, adhesive bonding, molding, and others. The internal and external tubes 12 and 14 are arranged for longitudinal axial movement relative to one another (except for their distal ends which are joined together).

Referring again to FIG. 2, steering tool 10 includes a handle 16 that has a tube manipulator 18 (also referred to as control knob 18) for causing longitudinal axial movement of one of the internal and external tubes 12 and 14 relative to one another so as to cause the distal portions of the tubes to bend or curve or otherwise deform. The same or another tube manipulator may be used to lock the tubes 12 and 14 of steering tool 10 completely or partially or not at all (i.e., unlocked so the tubes can move freely), as described in PCT Patent Application PCT/US2014/071075 or PCT/IB2017/051040. For example, control knob 18 may be a slidable knob that slides in a channel 20 and which may be lockable at different positions. In alternative embodiments, tube manipulator 18 may be a rotating element. The term “knob” encompasses any manipulative element, such us but not limited to, a knob, button, roller, switch and the like. For example, instead of being a mechanical knob, alternatively, the invention may be carried out with an electronic biasing system, in which case the control knob may be an electrical switch, such as a button or touchpad.

One or both of the internal and outer tubes may be formed with slots 24 (FIG. 3). The slots 24 may be phase-shifted with respect to one another (i.e., the slots on one tube may be phase-shifted with respect to one another and/or the slots of one tube may be phase-shifted with respect to the slots of the other tube). A phase shift manipulator (not shown, but described in PCT Patent Application PCT/IB2017/051040) may be used to apply torque to internal tube 12 or external tube 14, thereby changing the amount of phase shift between the tubes or between the slots, and thereby causing a twisted or spiral shape to the distal tips of the tubes. This provides the surgeon with limitless possibilities of shaping the distal tips to any desired, three-dimensional shape. The twisting torque also changes the stiffness of the bent portion of the tubes.

Handle 16 may include a medical device connector 22, such as a fluid connector, luer fitting and others.

As seen in FIG. 2, the added steering tool 10 passes through catheter 1 and extends to the distal end of the existing catheter 1. Catheter 1 has now been changed into a novel catheter with a distal end steering tool.

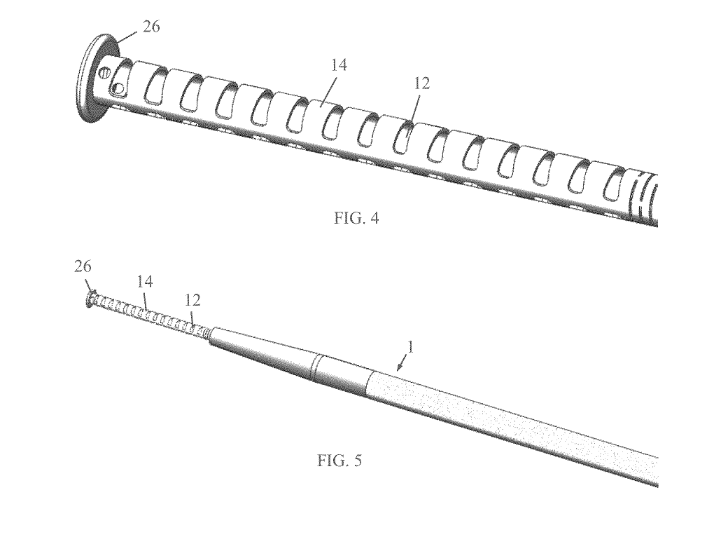

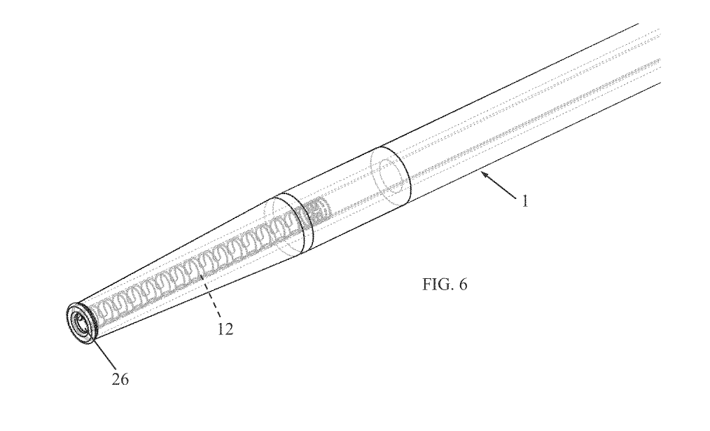

Reference is now made to FIGS. 4 and 5. In this embodiment, internal tube 12 may be provided with a stopper 26 or locker or any other coupling method to the distal end of the existing catheter 1. In other words, in one embodiment, there is no external tube 14; rather the existing catheter 1 serves as the external tube and the stopper 26, which abuts against the distal end of the catheter (as seen in FIG. 6), effectively couples the distal ends of the existing catheter 1 and the internal tube 12 so that longitudinal axial movement relative to one another causes the distal ends to bend as before. In such an embodiment, there is a cost saving by not using an external tube in addition to the catheter.

Alternatively, as shown in FIG. 5, stopper 26 may also be used in the embodiment that includes external tube 14. In such an embodiment, the stopper 26 is used to couple or join the distal ends of the internal and external tubes. The assembly of the internal and external tubes may extend distally from the distal end of the catheter.

In all embodiments, the internal tube 12 may be hollow or solid.

What is claimed is:

1. A method of changing a catheter into a distal steering tool catheter, comprising:
adding to an existing catheter a tube assembly that passes through said catheter, said tube assembly comprising an internal tube and an external tube whose distal ends are coupled to each other and which are arranged for longitudinal axial movement relative to one another; and
providing a tube manipulator coupled to said tube assembly, which is operative to cause relative axial movement of said internal and external tubes and bending of a distal portion of at least one of said internal and external tubes.

2. The method according to claim 1, wherein said external tube is different from said catheter.

3. The method according to claim 1, wherein said external tube is said catheter.

4. The method according to claim 1, further comprising using said tube manipulator to lock said tube assembly at a bent position.

5. An assembly comprising:
a tube assembly that passes through a catheter, said tube assembly comprising an internal tube and an external tube whose distal ends are coupled to each other and which are arranged for longitudinal axial movement relative to one another; and
a tube manipulator coupled to said tube assembly, which is operative to cause relative axial movement of said internal and external tubes and bending of a distal portion of at least one of said internal and external tubes.

6. The assembly according to claim 5, wherein said external tube is different from said catheter, and both said internal and external tubes pass through a distal end of said catheter.

7. The assembly according to claim 5, wherein said external tube is said catheter.

8. The assembly according to claim 5, wherein a stopper is coupled to a distal end of said internal tube.

9. The assembly according to claim 5, wherein said tube manipulator is operative to lock said tube assembly at a bent position.

10. The assembly according to claim 5, wherein said internal and external tubes abut against a distal end of said catheter.

11. The assembly according to claim 5, wherein said internal and external tubes pass through a distal end of said catheter.
